(19) United States
(12) Patent Application Publication
(10) Pub. No.: US 2008/0312579 A1
(43) Pub. Date: Dec. 18, 2008

(54) METHOD AND SYSTEM FOR MITIGATING CURRENT CONCENTRATION IN ELECTROKINETIC DRUG DELIVERY

(75) Inventors: Kuowei Chang, Lexington, MA (US); Dennis I. Goldberg, Sudbury, MA (US); Robert W. Etheredge, Natick, MA (US); Michael S. Barsness, Oxford, MA (US)

Correspondence Address: NIXON & VANDERHYE, PC, 901 NORTH GLEBE ROAD, 11TH FLOOR, ARLINGTON, VA, 22203, US

(73) Assignee: TRANSPORT PHARMACEUTICALS, INC., Framingham, MA (US)

(21) Appl. No.: 12/139,873

(22) Filed: Jun. 16, 2008

Related U.S. Application Data
(60) Provisional application No. 61/033,608, filed on Mar. 4, 2008.
(60) Provisional application No. 60/944,134, filed on Jun. 15, 2007.

(51) Int. Cl. A61N 1/30

(52) U.S. Cl. 604/20; 264/129

(57) ABSTRACT

An electrokinetic apparatus to apply medicament to a treatment site of a mammalian user, the apparatus including: a segmented active electrode; a medicament matrix having one side abutting the segmented active electrode and another side adapted to contact a surface of skin over the treatment site, wherein the matrix includes at least one current direction barrier suppressing transverse current flow through the matrix.

### RELATED APPLICATION

This application claims the benefit of U.S. Provisional Patent Application Ser. Nos. 60/944,134 filed Jun. 15, 2007, and 61/033,608, filed Mar. 4, 2008.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site on, in or under the skin of a human patient. In particular, this application is directed to electrokinetic delivery applicators for wide areas of skin to infuse medicament into a wide area treatment site and applicator for treatment sites having high resistances, such as a toenail or fingernail or area of hard skin.

Electrokinetic delivery of medicaments applies medication topically to the skin to reach a treatment site. One type of electrokinetic delivery mechanism is iontophoresis, which is the application of an electric current to the skin to enhance the permeability of the skin and thereby deliver ionic agents, e.g., ions of salts and other drugs, to the treatment site below the surface of the skin. Electrokinetic delivery methods include iontophoretic, transdermal, transmucosal cutaneous, electroosmosis, electroporation, and electromigration, any or all of which are more generally known as electrotransport, electromolecular transport or iontophoretic methods. These techniques are collectively referred to herein as electrokinetic delivery methods.

Electrokinetic delivery methods may be problematic when applied to, for example, large areas of skin, skin with highly variable impedances, or tissues with high intrinsic impedance such as toenails. Large skin treatment areas may be associated with skin conditions such as eczema, psoriasis and acne. To deliver medicament electrokinetically to a large skin treatment site, a relatively large medicament matrix is applied to the skin. A large electrical current is generally needed to electrokinetically drive sufficient medicament from the large area matrix into the skin. The matrix consists of a uniform solid phase within which is dispersed a uniform medicament formulation. Such a matrix is limited to delivering that medicament at one rate governed by the applied current from an electrode in general contact with the entire matrix. There is a need for a medicament matrix that is more responsive to the particular needs of tissues from one site to another beneath the matrix and that is capable of delivering one or more medicaments at various rates or dosages. Further, in lieu of a single electrode, an array of electrodes is superimposed on the matrix to establish electrical current through the medicament matrix and treatment site. Additionally, it is conventional for a counter electrode to be applied to the patient at a significant distance from the matrix. The current must flow through the patient from the delivery electrode adjacent the medicament matrix through the patient to the counter electrode. This resulting long current path through the skin may result in excessive voltage drop and poor control over spatial distribution of drug delivery.

Further, applying a large electrical current presents a risk of electrically sensitizing, irritating, or burning the skin, especially if the current is concentrated on a small skin area. A large area medicament matrix may be intended to contact a large area of skin. The uniformity of electrical impedance in a large area medicament matrix is not always maintainable due to factors such as patient movement, contact pressure, change of skin condition, development of skin lesion and liquid medicament migration. Area of low impedance may develop, and since current tends to follow the path of least resistance, the large current applied to the matrix as a whole may become concentrated on a localized area and may potentially cause tissue damage. In another example, the impedance of a toenail is high a relatively high voltage is applied to deliver medicament to the nail. The medicament matrix applied to the toenail may inadvertently touch the low impedance soft skin tissue next to the nail. There is a risk that a high current will flow and will be concentrated on the small area of soft skin that is inadvertently in contact with the medicament matrix. The high current concentration may cause a burn on the skin contact area adjacent the nail.

In other example, an electrokinetic applicator may be applied to a cold sore on a lip or the edge of the lip. The medicament matrix in the applicator is intended to contact the entire surface of the skin afflicted with the cold sore plus a surrounding area as a prophylactic measure. Due to the curvature of the lip and the edge of the lip, the applicator matrix may contact only a small skin area on the lip area rather than a larger skin area surrounding the cold sore. Current intended to be evenly distributed over the entire face on the applicator matrix may concentrate at the small skin contact area.

There is a long felt need for an electrokinetic device and method that delivers medicament and minimizes the potential for current concentration and associated burning of the skin contact area. In particular, there is a need for an electrokinetic device capable of delivering medicament using a high current applied to a large skin contact area, to a toenail or fingernail, or to a curved skin area without high density current being applied to soft tissue near the treatment site or a localized skin imperfection with relatively low impedance.

### SUMMARY OF INVENTION

An electrokinetic apparatus has been developed to apply medicament to a treatment site of a mammalian user, the apparatus including: a segmented active electrode; a medicament matrix having one side abutting the segmented active electrode and another side adapted to contact a surface of skin over the treatment site, wherein the matrix includes at least one current direction barrier suppressing lateral and transverse current flow through the matrix.

A method has been developed to electrokinetically deliver a medicament to a treatment site in a mammalian user, the method comprising: applying a first surface of a medicament matrix to a surface on the user; applying a segmented first electrode to a second surface of the medicament matrix; applying electrical current to an electrical current path extending through the first electrode segments, medicament matrix, at least partially through the user and to a second electrode; delivering medicament from the matrix into the treatment site by electrokinetically transporting the medicament along the current path, and blocking transverse current from one electrode segment of the first electrode to the surface on the user not aligned with the one electrode segment.

An electrokinetic apparatus has been developed to apply medicament to a treatment site of a mammalian user, the apparatus comprising: an active electrode including first and second active electrode segments; a counter electrode including first and second counter electrode segments; a medicament matrix having one side abutting the segments of the active electrode and another side adapted to contact a surface of the user over the treatment site, wherein the matrix includes at least one current direction barrier suppressing transverse current flow through the matrix; a first electrical circuit including the first active electrode segment and the first counter electrode segment, and a second electrical circuit including the second active electrode segment and the second counter electrode segment, wherein the first electrical circuit is galvanic-isolated from the second electrical circuit. This apparatus can also be expanded to include a multitude of active electrodes in addition to the first and second active electrodes.

An applicator panel has been developed for an electrokinetic delivery system including: an array of electrodes arranged on a flexible substrate and conductive pathways between each of the electrodes and a connector to receive a connection to a electronic control and power source circuit, wherein each electrode includes a center active electrode region and an outer return electrode region extending around the active electrode region, and at least one non-woven layer having a pattern of medicament cells and network of ribs between the cells, wherein the non-woven layer has a back surface laminated to the array of electrodes and the cells are each aligned with a respective one of the electrodes.

An applicator for an electrokinetic delivery system has been developed comprising: an array of electrodes arranged on a substrate and conductive pathways between each of the electrodes and a connector to receive a connection to a electronic control and power source circuit, wherein the electrodes are arranged in at least a first commonly connected group of electrodes and a second commonly connected group of electrodes; a first medicament layer having a pattern of first medicament cells, wherein the first medicament cells are aligned with the electrodes of the first commonly connected group of electrodes; a second medicament layer having a pattern of second medicament cells, wherein the second medicament cells are aligned with the electrodes of the second commonly connected group of electrodes, and an electronic controller controlling the electrokinetic delivery to a treatment site of a patient and underlying the first and second medicament layers, wherein medicament in the first medicament layer is delivered by electrically actuating the first commonly connected group of electrodes and medicament in the second medicament layer is delivered by electrically actuating the second commonly connected group of electrodes. The first and second groups of electrodes may or may not be of the same polarity. For example, an applicator pad with both anodic and cathodic electrodes would be well suited to deliver medicaments having both anionic and cationic actives to be delivered at the same time. Such an applicator having both anodic and cathodic electrodes may include a neutral polarity counterelectrode for each of the anodic and cathodic electrodes.

An applicator panel has been developed for an electrokinetic delivery system comprising: an array of electrodes arranged on a flexible substrate and conductive pathways between each of the electrodes and a connector to receive a connection to a electronic control and power source circuit; at least one non-woven layer having a pattern of medicament cells and ribs between the cells, wherein the cells are aligned with electrodes of the array; an adhesive matrix where the adhesive is aligned with the ribs, wherein the non-woven layer is sandwiched between the array of electrodes and the adhesive matrix.

A method has been developed to form an applicator panel for an electrokinetic delivery system comprising: embossing a pattern of medicament cells and ridges on a non-woven layer; infusing a medicament into the medicament cells; forming an electrode layer by applying an array of electrodes on a front side of an electrode layer substrate and forming an electrical distribution circuit on a back side of the electrode layer substrate, and securing the front side of the electrode layer substrate to a back side of the non-woven layer, wherein the electrodes are aligned with the medicament cells. The method may further include forming a first sub-layer and a second sub-layer of the non-woven layer, wherein each of the sub-layers includes a separate group of the medicament cells and the group of the medicament cells in the first sub-layer is non-overlapping with the group of the medicament cells in the second sub-layer.

An array of electrodes in an applicator panel has been developed for an electrokinetic delivery system including a medicament layer having an array of medicament cells, each of the electrodes comprising: a center electrode region aligned with one of the medicament cells; a non-conductive region surrounding the center electrode, and a neutral return electrode region encircling the annular adhesive seal. The center region in each of a first plurality of the electrodes is a cathodic center region coupled to a source of positive voltage and the center region in each of a second plurality of the electrodes is an anodic center region coupled to a source of negative voltage.

### DESCRIPTION OF THE DRAWINGS

FIG. 1 is a schematic diagram of an electrokinetic applicator including a multi-electrode active electrode, a medicament matrix and a cross-section of a cartridge housing the matrix and the active electrode.

FIG. 2 shows the cartridge in top down view to illustrate the multiple electrodes of the active electrode.

FIG. 3 is a schematic diagram of an electrokinetic applicator including a multi-electrode active electrode, a counter electrode, a medicament matrix and a cross-section of cartridge, wherein the cartridge and matrix is applied to a small area of the skin.

FIG. 4 is a schematic diagram of an electrokinetic applicator including a multi-electrode active electrode, a counter electrode, a medicament matrix and a cross-section of a cartridge, wherein the matrix includes a unidirectional foam, e.g., a honeycomb structure foam.

FIG. 5 is a schematic diagram of an electrokinetic applicator including a multi-electrode active electrode, a counter electrode, medicament matrix and a cross-section of a cartridge, wherein the cartridge and matrix may be applied to a small area of the skin.

FIG. 6 is a schematic diagram of an electrokinetic applicator including a multi-electrode active electrode, a counter electrode, a medicament matrix and a cross-section of a cartridge, wherein the cartridge and matrix may be applied to a small area of the skin.

FIG. 7 is a schematic diagram showing in side view a segmented active electrode and counter electrode applied to a toe or finger.

FIG. 8 is a schematic diagram of a galvanic-isolated current driver for each segmented active and counter electrode pair.

FIG. 9 is a schematic diagram of a galvanic-isolated current driver for each segmented active and counter electrode pair.

FIG. 10 is an exemplary circuit diagram of galvanic-isolated current driver.

FIG. 11 is a schematic diagram of an electrokinetic device having a wide-area applicator including a multi-electrode electrode and a medicament layer, coupled to an electronic power supply and controller.

FIG. 12 is a schematic diagram of a facemask electrokinetic device that includes a wide area applicator pad.

FIG. 13 is a schematic diagram showing in side view the applicator pad shown in FIG. 11 or 12.

FIG. 14 is an exploded view of the applicator pad shown in FIG. 12.

FIG. 15 is a back view of a medicament layer of the applicator pad.

FIG. 16 is a first embodiment of an array of electrodes for the electrode layer of the applicator pad.

FIG. 17 is a second embodiment of an array of electrode for the electrode layer of the applicator pad.

FIGS. 18 and 19 are schematic diagrams of an anodic and a cathodic electrode, respectively.

FIG. 20 is a schematic diagram of a front view of a medicament layer with multiple electrode panels, wherein each panel has sub-groups of separately controllable electrodes.

FIG. 21 is a schematic diagram of an electrode panel having four sub-groups of electrodes and each sub-group is separately connected to a current limiting circuit and the power source and controller.

FIG. 22 is an exploded view of multiple sub-layers of a medicament layer having multiple medicament sub-layers.

### DETAILED DESCRIPTION OF THE INVENTION

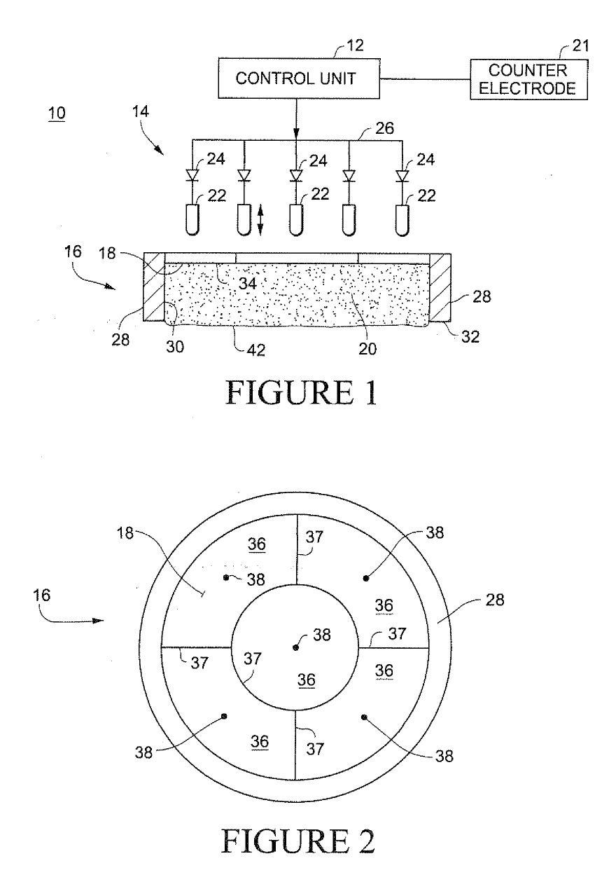

FIG. 1 is a schematic diagram of an electrokinetic medicament applicator 10 including a portable Electrokinetic Transdermal System (ETS) control unit 12, an active electrode power connector 14, a cartridge 16 that may be releasably attached to a housing for the ETS control unit, a segmented active electrode 18 and a matrix with medicament 20.

The ETS control unit 12 may be housed in a handheld device having an actuator switch to provide a manual trigger of the application of medicament by electrokinetic delivery. The ETS control unit may comprise a power system, such as batteries; a microcontroller for monitoring certain conditions, such as whether a valid cartridge is inserted in the device, and controlling the application of current to the active electrode, and conductive circuits connecting the power supply, microcontroller, actuator switch, active electrode and counter electrode. The ETS control unit, when actuated, applies current to each of the plurality of electrode segments of the active electrode. The current applied to the electrode segments may be, for example, on the order of 660 microAmps (uA). A current path includes the power connector 14, active electrode 18, medicament matrix 20 which is applied to the skin of a patient, e.g., a mammalian user, the patient, a counter electrode 21 that is applied to the patient, and the ETS control unit.

The active electrode power connector 14 may include a plurality contact pins 22, e.g., five pins, each having a current limiting device, such as a current limiting diodes (CLD) 24. The five diodes are arranged in electrical series with the contact pins. A conductive bus 26 provides a common connection between each pin 22 and diode 24 arrangements and the ETS control unit. The diodes each limit the current to a respective one of the contact pins 22 to, for example, 132 uA or about one fifth the total current applied by the entire electrode segments. The diodes may be selected to limit the current to each of the contact pins to a predetermined level, such as a current level determined by the total current applied to all electrodes divided by the number of contact pins 22.

The current limiting device 24 is preferably a simple, miniature current limiting device for each of the partition and/or sub-divided segments of the active electrode. The diodes 24 are one example of a current limiting device such as the generic current limiting diodes 1N5283 through 1N5314 and the CCLM0130 manufactured by Central Semiconductor Corporation. Other electronic circuit components that limit the current to each contact pin may be suitable. There are several types of current limiting diodes (CLD), such as a current regulator diode, constant current diode, and current limit diodes. Current regulating diodes regulate the current flowing through them to a maximum level and if current exceeds its current regulation point, it drops its terminal voltage. A constant current diode is similar to a junction field effect transistor (JFET) whose gate terminal is shorted to source. A constant current diode can automatically limit a current through a laser driver current limit diode over a wide range of power supply voltages. A laser driver current limit diode is a type of current limiting diode (CLD) that works on the principle of a quantum process whereby light is emitted due to transition of electrons from high-level to low-level energy states. Current limit diodes are employed to ensure that excessive current does not flow to any one of the active electrodes. The CLD is preferably arranged in the housing of the device (rather than in the cartridge), so that the CLD may be reused and is not discarded with the cartridge.

Current is distributed equally to each segment of the active electrode 18 in proportion to the number of electrodes and/or the size of the matrix corresponding to the corresponding electrode segment. The current is distributed, for example, by the bus 26 and the current limiting device 24 with each pogo pin 22. Because of the current limiting devices 22, the current flow through each segment 36 of the active electrode 18 is preferably limited and not excessive due to a small skin area or other condition that might lead to current concentration. The maximum current density applied to the matrix by each active electrode segment is generally equal to the current applied by the pogo pin 22 applied to the electrode segment divided by the area of the electrode segment.

The pins 22 may be spring biased, e.g., pogo pins, such that the pins are biased downward and can be deflected upwards, as shown by a double-headed arrow in FIG. 1. The active electrode power connector 14 may be mounted in a distal end of the housing for the handled device that includes the ETS control unit 12. The pins 22 may protrude from a recess in the distal end of the housing. The recess in the housing receives a connector of the cartridge. When the cartridge is mounted in the recess, the pins 22 are biased to establish electrical connection with electrode segments of an active electrode 18 in the cartridge 16. A conductive bus 26 provides a common connection between each pin 22 and diode 24 arrangements and the ETS control unit.

The cartridge 16 may have a generally cylindrical shape with an annular plastic wall 28 that defines a cylindrical recess 30 to receive the medicament matrix 20. The recess 30 may have an open face 32 which is applied to the surface of the skin or toenail to press the medicament matrix against the skin or nail. The cartridge may alternatively be embodied as an array of cartridges that are applied to various locations on the skin or nail.

FIG. 2 shows the cartridge 16 as a cross-section. The cross section exposes a back surface of the segmented active electrode 18. The electrode 18, in one embodiment, is a thin laminated disc including metal segments 36 arranged on a substrate. The active electrode is in electrical contact with the medicament matrix 20. The substrate may be applied to the backside of the segments. Each electrode segment 36 is electrically isolated from the other active electrode segments, such as by a gap 37 between the electrode segments. The gap may be a dielectric material such as air, plastic or paper. The back surface of each electrode segment 36 includes a contact pad 38, e.g., a portion of the segment exposed through the substrate, which receives a distal end of a pogo pin. The contact pad 38 is exposed through an aperture in the cartridge to receive the pins.

The active electrode 18 may be mounted on a surface 34 (FIG. 1) of the cartridge recess 30 that is opposite to the open face. The active electrode 18 has a first surface in contact with the medicament matrix 20 and an opposite surface abutting cartridge surface 34 and positioned to receive the pogo pins 22, when the cartridge is mounted in the device housing. The medicament matrix 20 may be a cylindrical disc mounted in the recess 30 of the cartridge. A backside of the matrix is in electrical contact with the electrode segments 36 of the active electrode 14. A front side 42 of the matrix extends slightly from the face 32 of the cartridge. The front side of the matrix is exposed and is to be placed against the skin or a nail of a toe or finger. A removable foil lid, not shown, may cover the front side of the matrix and seal the matrix in the cartridge. The lid is removed prior to the application of the matrix to the skin or nail.

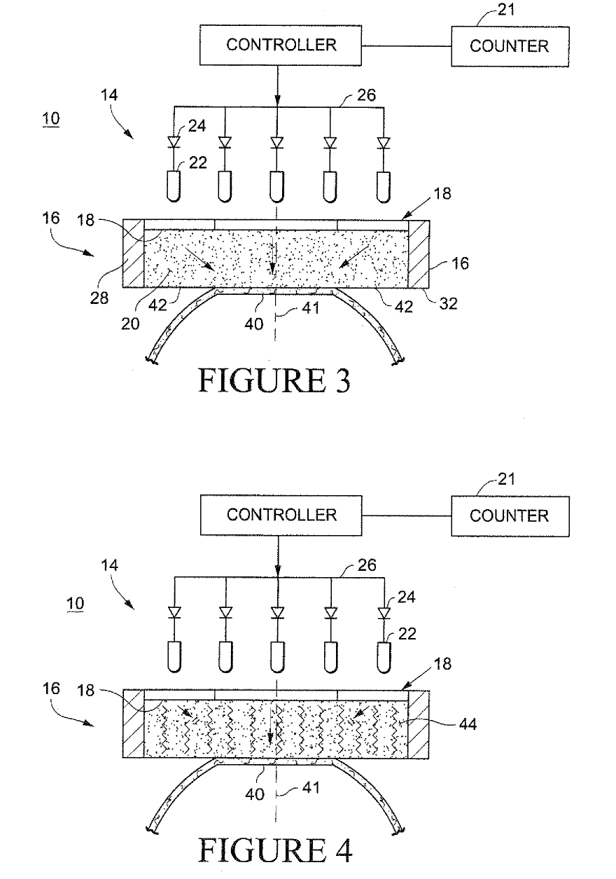

FIG. 3 is a schematic diagram of an electrokinetic applicator 10 including a multi-electrode active electrode 18, a medicament matrix 20 and a cartridge 16, wherein the cartridge and matrix is applied to a small area 40 of the skin. The small skin contact area 40 may be, for example, a cold sore which is on a curve lip. When the cartridge and matrix are applied to the small cold sore 40, only a portion of the open face 42 of the medicament matrix 20 is in contact with the skin surface. The applicator 10 is designed based on an assumption that the entire open face 42 of the matrix 20 is to be in contact with an area of the skin that is at least as large as the face 42 of the matrix. In particular, the electric current applied by the active matrix 18 is set at a level assuming that the skin contact area covers the entire area of the face 42 of the matrix.

If the skin contact area 40 is smaller than the matrix face 42, the current may become concentrated on the small skin contact area 40 as is illustrated by the arrows in FIG. 3. The limited skin contact area may receive current not only from a central active electrode segment, but also from surrounding active electrode segments which may not be axially aligned with the skin contact area. If the matrix 20 is an isotropic foam or other material that allows current to flow at an angle to the cartridge axis 41, current from active electrode segments that are not axially aligned with the skin contact area can tunnel at an angle to the axis through the matrix, to the small skin contact area 40. The electrical surface potential of each of the active electrode segments will attempt to self-adjust the voltage difference between the skin contact area and each of the active electrode segments. But for the current limiting devices 22, the self-adjustment may result in excessive voltages and/or currents to one or more of the active electrode segments. The current limiting devices 22 help prevent excessive current at any of the active electrode segments 18. However, transverse current tunneling through the matrix may funnel extra current to the small skin contact area 40 and may result in current concentration even with segmented electrodes and current limiting devices.

FIG. 4 is a schematic diagram of an electrokinetic applicator 10 including a multi-electrode active electrode 18, a medicament matrix 44 and a cartridge 16, wherein the cartridge and matrix may be applied to a small area 40 of the skin. The matrix 44 includes a foam having internal structure that prevents lateral current flow transverse to the axis 41 of the cartridge. The foam structure may include dielectric fibers or matrix features generally aligned with the axis 41 of the cartridge.

The foam structure of the matrix 44 may be provided by specialty foams that mimics a honeycomb structure and allows ion to flow only in one direction, i.e., parallel to axis 41. The structure of the matrix 44 effectively blocks transversal ionic flow and prevents current tunneling from neighboring segments of the active electrode. Current concentration is effectively avoided by the use of a current directional foam (or other medicament matrix that prevents lateral current flow transverse to the cartridge axis) in combination with a segmented electrode having current limiting devices. The foam structure minimizing transverse current flow augments the effect of current limiting diodes connected to a multi-electrode matrix and the intrinsic resistance to lateral current flow of the medicament and/or skin.

Unidirectional-aligned open cell foams and films suitable for use as a medicament matrix 44 are well known and include the Celgard™ microporous polypropylene film sold by Hoechst-Celanese Plastic Company of Newark, N.J. and is a flexible microporous film with a unidirectional structure. Unidirectional aligned polypropylene open cell foams have also been reported by BASF (Journal of Material.Science 41, 2006). These foam matrix materials may be applied as pharmaceutical grade, unidirectional current foams for use as medicament matrix.

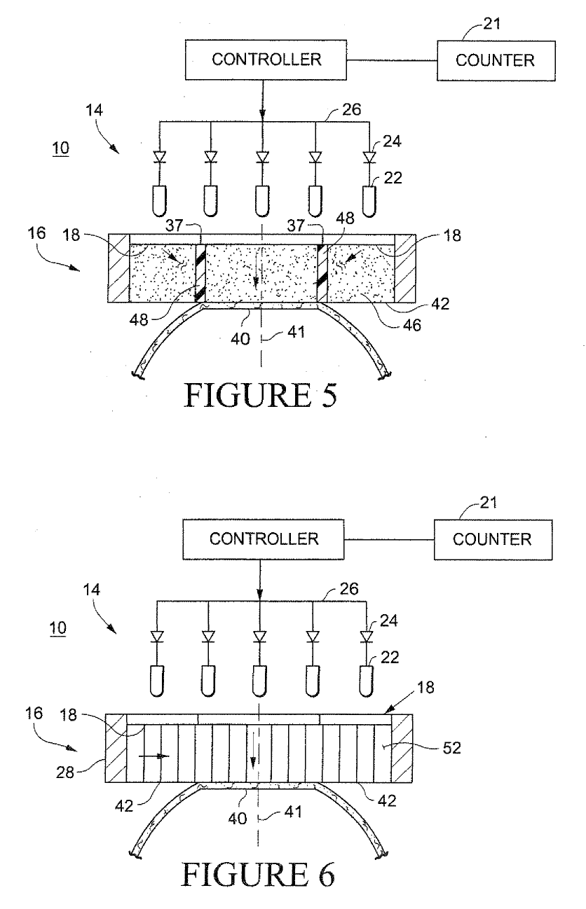

FIG. 5 is a schematic diagram of an electrokinetic applicator 10 including a multi-electrode active electrode 18, a medicament matrix 46 and a cartridge 16, wherein the cartridge and matrix may be applied to a small area 40 of the skin. The medicament matrix prevents transverse ionic current flow between an active electrode segment and a skin contact area that is not axially aligned with the segment by dielectric barrier walls 48. The barrier walls 48 extend the depth of the medicament matrix 46 and, particularly, extend from the active electrode 18 to the open front face 42 of the matrix. The barrier wall may be formed of, for example, medical grade Silastic silicone rubber separators. The barrier walls may be arranged along the separation gaps between the segments of the active electrode.

The barrier walls 48 in the medicament matrix are aligned with axis of the cartridge. The barrier walls are dielectric and may comprise silicone rubber, air, or other dielectric material. The barrier walls are preferably aligned with the gaps 37 between the segments 36 (FIG. 2) of the active electrode 18. Current transverse to the axis 41 is blocked by the barrier walls. Accordingly, current from one electrode segment does not tunnel through the matrix to a skin contact area 40 below another electrode segment.

FIG. 6 is a schematic diagram of an electrokinetic applicator 10 including a multi-electrode active electrode 18, a medicament matrix 52 and a cartridge 16, wherein the cartridge and matrix may be applied to a small area 40 of the skin. The medicament matrix 52 is formed of a fine silicone rubber and has a honeycomb structure. The honeycomb structure segments the medicament in electrically isolated columns, where the columns each extend from an active electrode to the front face 42 of the matrix. The electrically isolated columns in the matrix prevent current flow at a substantial angle to the axis 41 of the cartridge. In particular, the columns prevent current from an active electrode segment to flow to a skin contact area not axially aligned with that electrode segment. Medicament is contained in the columns of the honeycomb matrix 52. When current is applied to the active electrode, the medicament is delivered electrokinetically to the treatment site below the surface of the skin contact area 40. Since a thin film of conductive medicinal solution may exist on skin surface 40, an adhesive, such as Dow Corning 2013 pressure sensitive adhesive, may be applied to distal end of the silicone rubber honeycomb structure near the front face 42. This adhesive forms a tacky end surface adhering to the skin, serving as an effective barrier to any lateral current leakage along the skin surface.

The use of strategically positioned segmented electrodes, current limiting diodes, and unidirectional foam material (such as with honeycomb-like structure and as shown in FIGS. 3 to 6) may be used to alleviate or eliminate current concentration in a skin or nail treatment site. The use of segmented electrodes, current limiting diodes and unidirectional foam, reduces the risk of detrimental current concentration on a small skin contact area. The exemplary embodiments shown in FIGS. 3 to 6 offer an advantageous means of preventing current tunneling and current concentration in a segmented electrode implementation. These embodiments are particularly effective for applicators having a cartridge with a medicament matrix.

Onychomycosis is the invasion of a toenail plate by a dermatophyte, yeast or nondermatophyte mould. The medicament may be used to treat, for example, onychomycosis, which is a fungal invasion of the nail. The fungal infection may be due to a dermatophyte, yeast, or nondermatophyte mould. The medicament is intended to destroy the fungus or at least cause the fungal invasion to subside. For effective onychomycosis treatment, it is desirable to have the medicine, e.g., Terbinafine hydrochloride or Fluconazole, penetrates the nail and saturates the surrounding tissues. In view of the high electrical impedance of the nail plate, a high voltage, such as over one hundred volts, may be required to electrokinetically deliver the medicament to surrounding tissues in onychomycosis treatment. The toenail involved in onychomycosis treatment is often small and the matrix applied to the nail may touch the soft tissue adjacent the nail. Current concentration may occur due to the high current applied by the active electrode, especially if a portion of the matrix is in contact with the soft tissue adjacent the nail. Since current will seek the path of least resistance, current may traverse the medicament matrix to flow to the soft tissue. In this manner, severe current concentration can occur at the soft tissue in contact with the matrix.

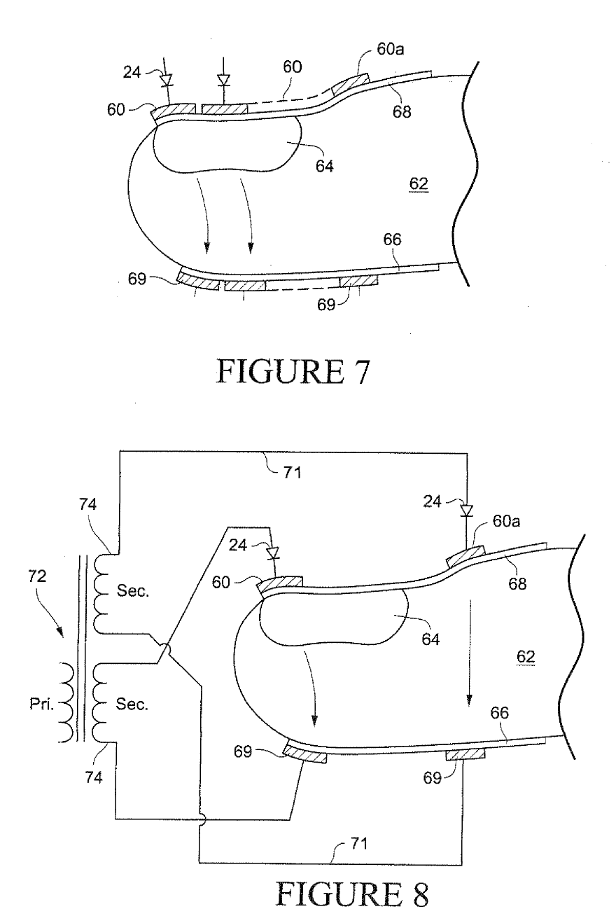

FIG. 7 is a schematic diagram showing in side view a segmented active electrode 60 and counter electrode 66 applied to a toe or finger 62 and specifically to the nail 64 of the toe or finger. The counter electrode 66 may comprise a layer of electrolyte material and segmented metallic counter electrode segments 69 that are each connected to a separate conductive wire. The active electrode 60 and counter electrode 66 may be each segmented. The segments may be arranged in pairs of active and counter electrode pairs.

The electrolytic layer of the counter electrode 66 may be a strip having an adhesive surface that adheres to the pad of the toe or finger. The active electrode segments 60 may be mounted on the medicament matrix 68, e.g., a porous sheet with an adhesive surface that adheres to the nail and upper skin tissue of the toe or finger. Each electrode segment 60 is in series with a current limiting device, e.g., a current limiting diode 24. The medicament matrix sheet 68 for onychomycosis treatment may include a thin hydrogel layer which may be a web applied to the nail. The medicament matrix 68 may extend beyond the nail and cover portions of the soft tissue of the toe or finger, as does the matrix below active electrode segment 60a.

The counter electrode segments 69 are each electrically coupled to a corresponding segment of the active electrode 60. An electrical circuit 71 is formed for each pair of active and counter electrode segments, wherein the circuit couples the pair to a power source and isolates the pair from current in the circuit 71 for other pairs of active and counter electrode segments. To safeguard against current concentration, each pair of active and counter electrode segments may be electrically isolated from the other pairs of electrode segments.

FIG. 8 is a schematic diagram of a galvanic-isolated current driver 72 for each segmented active and counter electrode pair 60, 69. The current driver includes a transformer having primary winding(s) coupled to a power supply (not show). A secondary winding 74 is provided for each of the active and counter electrode pairs. A circuit is formed by each pair of active and counter electrode segments and the secondary winding electrically coupled to the pair. Because of galvanic isolation, the treatment current delivered to each active electrode segment 60, 60a returns through the finger to the counter electrode segment 69 that is electrically coupled to the corresponding active electrode segment and secondary winding. Current from one pair of active and counter electrodes should not flow to another pair of active and counter electrodes. Miniature transformer 72 may be arranged to supply current for each of the circuits 71.

The current driver 72 may be a conventional transformer in which secondary voltage is determined by the ratio of primary to secondary transformer turns and controlled by a primary side oscillator driver power supply circuit (not shown). The current limiting diode 24 on each of the active electrode segments controls the current level applied by each active electrode segment to the matrix 68. The electrical circuit consisting of secondary winding 74, current diode 24, active and counter electrode segments 60 and 69, is intentionally simplified for clarity. Other active circuit elements can also be included to add more sophistications and features, if desired. Providing isolated pairs of electrode segments, minimizes current concentration in the presence of especially disparity in impedances between cuticle, nail folds, and nail plate. Use of segmented electrode pairs addresses the current concentration issue on the active and counter electrode side.

Using segmented active/counter electrodes for current concentration abatement has other benefits. By measuring the voltage drop across each electrode segment pair 60 and 69, and by dividing it with the current magnitude, the resistance of the current path can be determined and monitored either by analog or digital circuitry, or by a simple microprocessor operating within the same galvanic-isolated power loop.

In conducting electrokinetic transdermal drug delivery and onychomycosis treatment, an abnormal breach in the skin or nail may form such as a localized lesion, sore, pin hole or crack may develop in the skin or nail during treatment. Such an abnormal breach of the skin or nail barrier property can lead to a substantial reduction of local tissue impedance. Although the current of each segmented electrode pair is limited by the current limiting diode 24, the current density at the localized lesion can still be high enough to cause discomfort, burn, and tissue damage. This sudden impedance reduction can easily be detected by the microprocessor, and the current can either be reduced or switched off totally.

Since resistance of the current path for each individual segmented electrode pair can be measured, also since the current for each electrode pair can be separately controlled, a method of area mapping and current control for large skin area treatment is devised. As an exemplary case, consider large area facial eczema treatment using segmented electrodes and unidirectional medicinal foam matrix. Although the eczema afflicted area and topology may vary from patient to patient, a general purpose large area electrode containing a multitude of segmented active electrode can be used. In treatment, the medicament matrix is trimmed to cover the afflicted area. The flexible, segmented electrode sheet, being over-sized by design, is placed over the entire afflicted area. Since the resistance of each segmented pair can be measured, the afflicted area is identified as being with normal medicinal matrix resistance, whereas the un-afflicted area exhibits high resistance. Thus the afflicted area can be precisely mapped and treatment currents are switched on and delivered only to electrode pairs associated with the active area. There is no voltage being biased across un-used electrode pairs and there is no current flow to un-treated area. Impedance mapping allows for activation of active segments and de-activation of idle segments. It also allows for use of a universal active electrode design suitable for all patients.

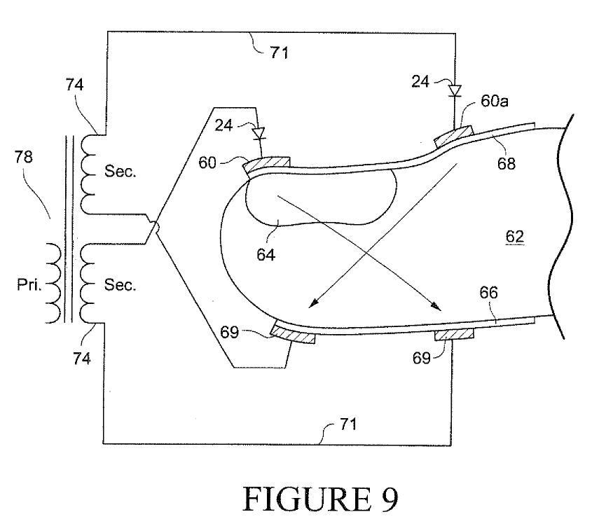

FIG. 9 is a schematic diagram of a galvanic-isolated current driver 78 for each segmented active and counter electrode pair 60, 69. The drive 78 is a transformer with multiple secondary windings 74, wherein each secondary winding is connected to respective pair of active and counter electrode segments 60, 69. The various segmented electrode pairs may be arranged in juxtaposition as shown in FIG. 8 and indicated by the parallel current arrows. Instead, the electrode pairs may include counter electrode segments transverse to the corresponding active electrode segment, as is shown in FIG. 9 by the crossing current arrows. The galvanic isolated current loops shown in FIGS. 8 and 9 are a safeguard against current concentration in nail fungus treatment. An optional and additional safeguard would be to use unidirectional foams or films for the medicinal matrix as disclosed in connection with FIGS. 3 to 6.

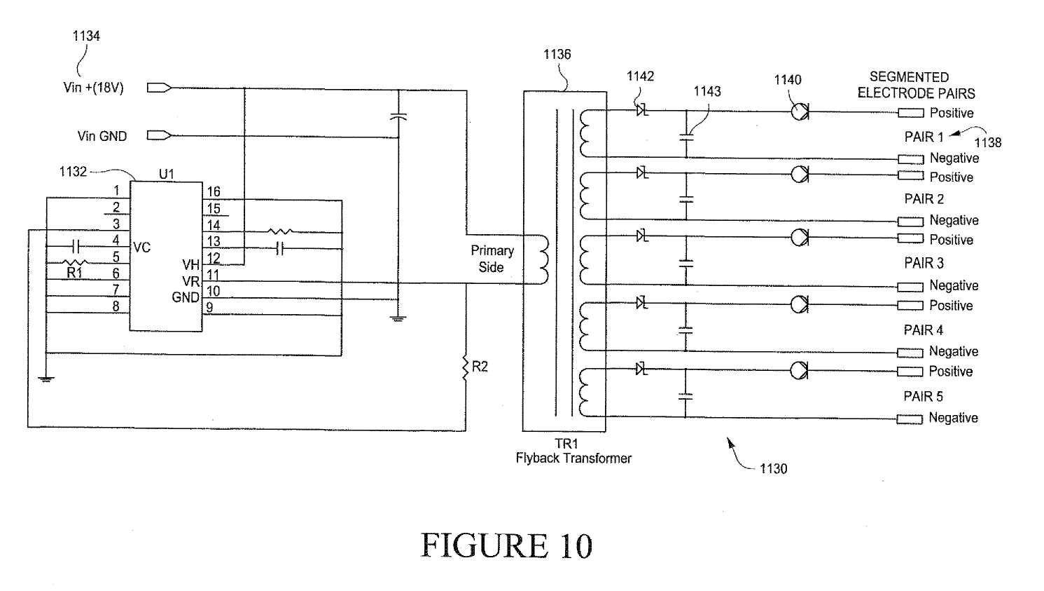

FIG. 10 is an exemplary circuit diagram of a galvanic-isolated current driver 1130. This current driver includes a galvanic isolation circuit that includes a flyback switching regulator 1136, e.g., a flyback transformer, operating in current mode topology where the secondary voltage is controlled by a microcontroller 1132 on the primary side of the flyback transformer. The flyback transformer can operate as a step-up booster transformer to generate very high voltages especially with a high secondary-to-primary turns ratio. The microcontroller 1132 regulates the current applied to the primary side of the flyback transformer. For example, the value of resistor (R2) or the ratio of resistors (R1/R2) sets the maximum voltage applied to the primary winding and hence the maximum treatment voltage applied to the secondary windings of the transformer and to the electrodes 1138. The microcontroller controls charging and shorting the primary coil to pump energy to the secondary side and into capacitors 1143 in the circuits for each of the segmented electrodes 1138. The energy pump produces a current spike that the flyback transformer 1136 steps up and generates a high voltage to be rectified 1142 into a high DC voltage. The resistors R1 and R2 may be dynamically controlled to ramp up and ramp down the voltage applied to the primary windings.

Within the loop of each pair of segmented electrodes 1138, current flows from the active (positive) to the counter (negative) electrode through a medicament matrix, the treatment site and the body of the mammalian user. The current magnitude in each current loop is limited to a value controlled by the current limiting diode 1140 irrespective of the nail and tissue impedance. Although a high DC voltage is generated within each loop, this voltage is self regulating and it will drop entirely across the current limiting diode 1140, nail plate and the toe. Each current loop for each electrode pair maintains a pre-set current which is galvanic-isolated in so far as the coils are isolated. Because a sufficient amount of energy is transferred to the secondary side of the flyback transformer 1136 to obtain a sufficient high DC voltage, the full current allowed by each current diode is maintained. The nail and toe are effectively a short-circuit down stream from the current diode operating in the “limiting” mode.

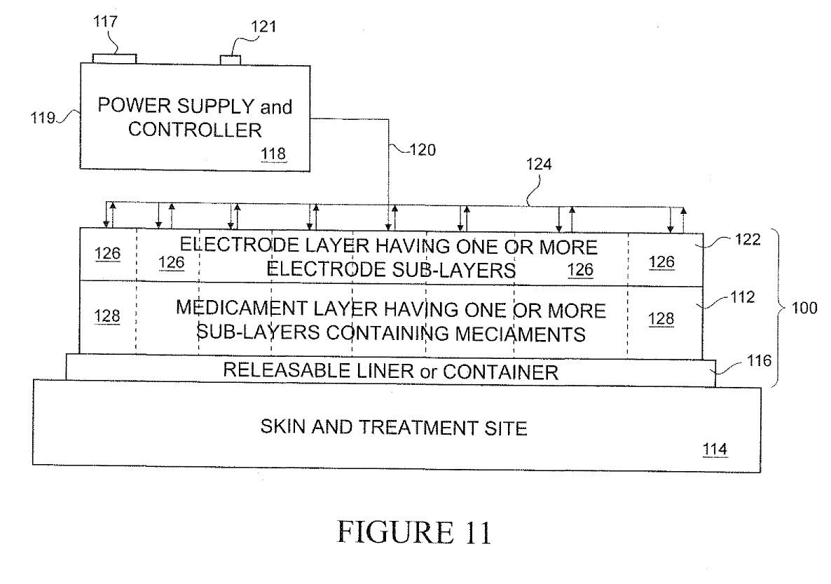

FIG. 11 is a schematic view of a multi-channel iontophoretic wide-area applicator panel 100 having a medicament layer 112 formed of one or more non-woven sheets. The applicator panel may be configured, for example, as a flexible panel or pad and be incorporated in an adhesive coated pad, facemask, glove and other medicament applicator. The applicator panel is placed on the skin 114 of a patient on or over a treatment site. Before placing the applicator panel on the skin, a releasable liner 116 may be removed from a front face of the applicator panel or the applicator panel may be removed from a sealed container.

The applicator panel is connected to a power source and computer controller 118 that may be mounted on the applicator panel or attached by electrical wires 120 to an electrode layer 122 on a side of the medicament layer opposite to the skin. Electrical current through the wires 120 from the power supply and controller to an electrical distribution circuit 124 that directs current to and from individual electrodes 126. The electrical power may be delivered through separate electric current channels to each electrode 126 such that the amount of current applied to each electrode may be separately controlled by the controller 18 or other circuits associated with the distribution circuit 124.

Each electrode 126 may include an active and neutral electrical terminal. There is a neutral electrode that is unique and local to each active electrode. The current path between the active and neutral electrical terminals of each electrode passes through the medicament layer and the treatment site. Accordingly, electrical power passing through each electrode causes medicament in the medicament layer to be delivered to the treatment site as the current in the power passes between the active and neutral terminals of the electrode.

The power supply may include batteries contained in a housing with the controller or may include an adapter that plugs into a conventional electrical current supply, such as an electrical wall socket. The housing 119 for the power supply and controller portion may be releasably coupled to the applicator panel 100, wherein the connection includes the wires 120 for providing electrical power and control signals between the housing and the applicator panel. The housing may also include user interface devices, such as an control switch(es) 121 and a liquid crystal (LCD) display. The control switch(es) 121 allows the user to input data and control signals into the controller, such as a medicament delivery signal or a code from a drug prescription order to indicate to the controller an amount, delivery rate and composition of medicament to be delivered to the patient. The display 117 may show to the user data generated by the controller identifying the medicament to be dispensed, application instructions, such as a location on the body to which the applicator panel is to be applied and a time period that the applicator panel is to remain on the body.

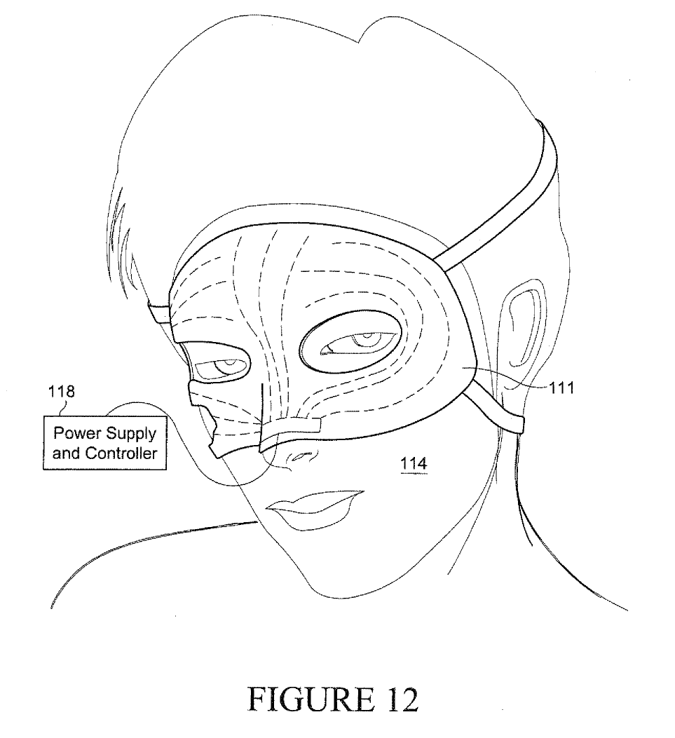

FIG. 12 is a perspective view of an exemplary embodiment of a face mask applicator panel 111 having components substantially the same as the components of the applicator panel 100 shown in FIG. 11. The facemask applicator panel 111 may be strapped to the head of a patient. The applicator panel has a wide-area interior surface that conforms to the 114 of the face of a patient. The total area of the face mask applicator in contact with the skin may be generally 12 cm (centimeters) by 10 cm and shaped to conform to the face. The face mask may be divided into sub-sections of roughly three cm² per sub-section. Within each sub-section, the medicament cells may be grouped into four separate groups each having a corresponding group of electrodes. By controlling separately the current to each group of electrodes and cells, the amount or type of medicament to be delivered may be selected or adjusted to the particular patient receiving the medicament. Each group of the medicament cells may cover a skin area of approximately 0.78 cm². The total face mask area of 120 cm² may be covered by approximately 40 sub-panels of electrodes, each divided into four zones of electrodes. The controller may separately control each of the 160 zones of electrodes.

Medicament stored in the cells 128 (FIG. 11) in the medicament layer 112 of the facemask applicator panel 111 is delivered to the facial skin by applying electrical current through wires and electrodes (represented by dotted lines in FIG. 12) in the facemask. The user may activate the application of electrical current by pressing a start button on the housing for the power supply and controller 118. The facemask applicator panel may be used to a treatment for facial wrinkles such as by electrokinetically delivering a modulator of collagen deposition, an organic nitrate, e.g., gallium nitrate. Similarly, the facemask applicator panel 111 may be used to apply metronidazole for rosecea, which is a facial skin condition.

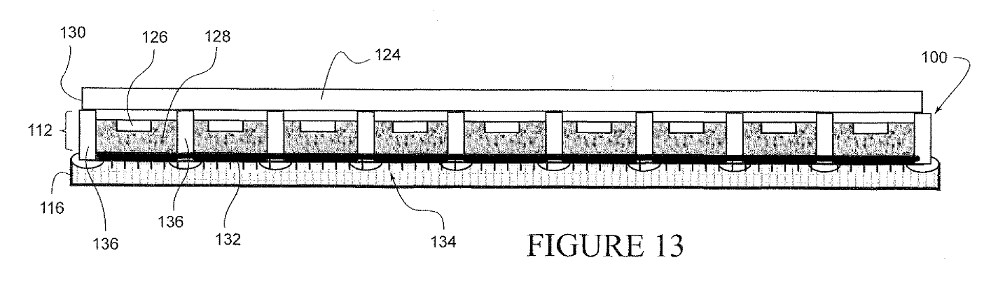

FIG. 13 is an enlarged side view of the applicator panel 100 having a medicament layer 112 comprising an array of medicament cells 128, an electrode 126 for each cell, an electrical distribution circuit 124 for the electrodes, and a release liner 116. The applicator panel 100 may be formed of laminated layers including the medicament layer 112, electrical distribution circuit and electrode layer 130, and release liner layer 116. The applicator panel may be, for example, a two inch by two inch square panel that is applied with adhesive or configured as a facemask, embedded in a glove or other wearable device. Further, the applicator panel may be shaped by the patient, such as cut to fit on a toenail, curved and folded to fit onto an elbow, or otherwise trimmed and shaped to conform to the skin at a treatment surface.

The applicator panel 100 may be flexible to conform to the body in a manner that is comfortable to the user. The user may self-administer the medicament by applying the applicator panel to his or her skin and over the treatment site, which is the portion of the skin or body to receive the medicament. Before applying the applicator panel, the release liner is removed to expose the front surface 132 of the medicament layer 112. The release liner may be a sheet having a layer of impervious plastic to prevent seepage of the medicament while the applicator panel is in storage and an adhesive to adhere to the front surface of the medicament layer. The exposed front surface 132 of the medicament layer may include a microporation layer 134, such as an array of micro-needles that perforate an upper layer of the skin as the applicator panel is applied to the body. The micro-needles assist in the delivery of medicament to the treatment site. The microporation layer porates the stratum corneum of the skin, including a layer that applies alternating current (AC) or direct current (DC) electroporation, an ultrasound layer, a layer applying RF ablation from small RF electrodes, laser, and other mechanical and electromagnetic means that provide poration of the stratum corneum.

The applicator panel 100 may be used to electrokinetically transport a medicament into the skin and is particularly useful for applying medicament over large wide areas of an individual's face and body. The applicator panel 100 may be used to treat various dermatological conditions, such as eczema, psoriasis, acne, boils, and blemishes, provide anesthesia, or to provide dermal exfoliation. In general, iontophoresis is well suited to the targeted dermal delivery of medicaments, e.g., pharmaceutical drugs. The application of an electric field to the skin underlying the applicator panel enhances the ability of various ionic agents in the medicament or its transport materials to penetrate the skin barrier. Medicament from the medicament cells is delivered to the treatment site by forming an electrical path from the medicament cells, to the treatment site and to electrodes connected to a power supply. Electric current flows from the power supply to an electrode, into the medicament cells, to the skin and treatment site, to another electrode in contact with the skin and back to the power source. As the current flows from the medicament cells, through the skin and into the treatment site, medicament is delivered from the cells to the treatment site.

The medicament layer 112 may include an array of medicament cells 128, such as a honeycomb arrangement of cells. These cells may be each independently controlled or controlled in subset cell groups by the controller. Medicament, and optionally hydration and ion transport materials used in combination with the medicament to assist in electrokinetically delivering the medicament, may be stored in the cells by infusing the medicament in cells, such as the fibers that form the cells and medicament layer. The cells may be arranged in a two-dimensional array in the medicament layer. In this exemplary cellular array, each cell may have a dimension of approximately 2 mm (millimeters) for each edge. The cells may be shaped as squares (2 mm×2 mm), circular discs (2 mm diameter) or other shapes. The cells may be arranged in arrays formed of rows and columns in the layer 112, rows in which the cells are staggered with respect to cells in adjacent rows, concentric circles or in other arrangements.

The cells may be spaced apart from each other by about 2 mm. A network of ribs, e.g., ridges, 136 in the medicament layer 112 may separate the cells and provide a substrate for an adhesive to attach to the release liner and later to the skin. The adhesive may be a zone-coated adhesive applied in a pattern to conform to the network of ribs. The adhesive may form a seal between each cell and the skin and thereby assist in isolating the cell from each other when the applicator is applied to the skin. The network of ribs may be a network of rows and columns of material forming the medicament layer that extend between the cells. Further, the ribs may be embodied as ridges that protrude from the back side of the medicament layer that faces the electrode layer. The front side of the medicament layer may be relatively smooth. In one example, an array of cells 128 separated by a network of ribs 136 provide an open area (between the ribs) of about thirty percent (30%) of the area of the front face 132 of the medicament layer 112 is open area of the cells for drug delivery. The medicament layer(s) may be formed with a conventional flexible non-woven web of fibrous material, such as polyolefins, polyester, nylon, cotton or other synthetic or natural fibers and blends thereof. The layer 112 may have a material property of about 100 g/m² (grams per meter squared) basis weight and a thickness of approximately 1.5 mm. By way of example, a one centimeter squared (1.0 cm²) pad for an applicator panel would provide about 0.3 cm² open area of cells for medicament delivery and a volume of about 45 ul (micro liters). The actual liquid volume of medicament delivered by the cells may be about 25 ul.

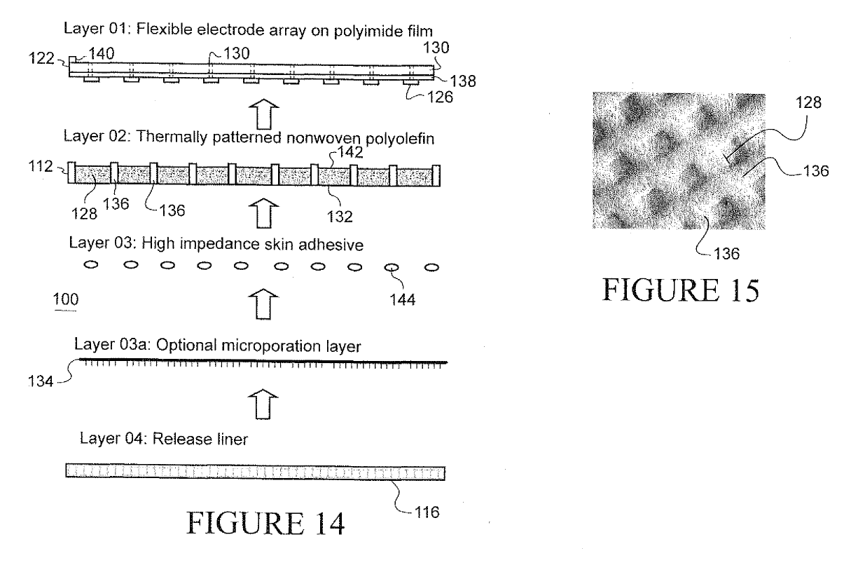

FIG. 14 is a diagram showing side views of the various layers and assembly of an applicator panel 100. The applicator panel comprises several layers that combine to create a flexible, skin-adherent, multi-channel iontophoretic drug delivery device. The applicator panel is formed by laminating flexible layers including: an electrode array layer 122, one or more patterned non-woven layers 112 with medicament cells 128, an adhesive layer 122, a microporation layer 134 and a release liner 116. The layers in the medicament layer 112 may include separate layers for each of one or more groups of medicament sub-layers.

The electrode layer 122 (Layer 01) may be formed of a flexible substrate 138. The electrode layer is a compliant electrode layer that provides current to discrete and isolated electrodes in the layer. The electrode layer may include as a polyimide film, such as Kapton® offered by DuPont or an equivalent high temperature resistant film. The polyimide film may be perforated to provide multi-axial electrical connections through the substrate between each of the electrodes, on one side of the film, to a distribution circuit, on an opposite side of the film. Alternately, a conductive distribution circuit may be screen printed in a desired pattern on flexible substrates such as textiles, nonwovens and films. By way of example, the film surface around each electrode in the electrode layer may be bordered by a heat seal layer comprised of ethylene vinylacetate, ethylene acrylic acid, or equivalent to which layer is thermally bonded to the electrode layer. On side of the film or substrate opposite to the electrodes, an array of conductive lines may be etched, printed or otherwise generated to form the electrical distribution array 130. On the opposite side of the substrate is formed the array of individual electrodes 126, wherein electrical contacts extend through the substrate to provide conductive contacts between the electrodes and the distribution array.

The flexible substrate 138 may have an electrical distribution array 130 on one side of the substrate and electrodes 126 on the other side of the substrate. The distribution array 130 includes electrical connection contacts 140 to connect the array to the wires 130 leading to the power source and computer controller.

The medicament layer 112 (Layer 02) is attached to the electrode layer 122 by, for example, an adhesive. The medicament layer may be attached to the electrode layer such that the electrodes 126 are aligned with the cells 128 of the medicament layer 112. The medicament layer may be formed of a polyolefin non-woven material that has been thermally patterned through a conventional point-bonding process to create an array of medicament cells 128 and a network of ribs 136. The cells form discrete reservoirs containing medical drug formulations for delivery to the treatment site at the skin. The medicament layer may also be embossed to form the cells 128 and the ribs 136 between the cells.

FIG. 15 is a back view of the medicament layer 112 that has been embossed to form the ridges 36 in a cross-hatch pattern and cells 128 between the ridges. The back view shows the back surface 142 of the medicament layer in which the ridges are raised. The front surface 132 of the medicament layer 112 may be relatively flat to facilitate the application of the cells 128 directly to the skin and treatment site. The ridges extend to the front surface, but may not necessarily protrude from the front surface.

An adhesive 144 (Layer 03) may be applied to the front surface 132 of the medicament layer to secure to the layer the release liner (Layer 04) 116 or optional microporation layer 134 (Layer 03a), e.g., microneedles. The adhesive may be applied to the portion of the front surface 132 corresponding to the ribs 136 to avoid the cells and potentially blocking the flow of the medicament from the cells to the skin. The adhesive may be a patterned, hypoallergenic adhesive applied through a hot-melt process to the front surface of the medicament layer and particularly to the ribs 136 between the cells of the front surface of the medicament layer. The adhesive 144 may be a high impedance material and, as such, may be used to electrically isolate the cells from one another. Further, the adhesive 144 may serve as a gasket seal between the skin and the cells. The adhesive may be a skin adhesive of variable tack and adhesion may be chosen depending upon the particular needs of the dermal substrate.

The microporation layer 134 is porous and may be applied directly to the front surface of the medicament layer. The medicament flows from the cells, through the microporation layer, e.g., a network of microneedles, and into the skin. The microporation layer 34 may be laminated to the front surface of the medicament layer. The microporation layer 134 may contain an array of microneedles or other microstructures that act to breach the stratum-corneum barrier of the skin and allow greater through-put of iontophoretically delivered actives. The microporation layer may be thermally fused to the overlying medicament layer, e.g., a polyolefin non-woven layer, to create a hermetic seal between the cells.

The layers of the applicator panel 100 are laminated together preferably in the sequence shown in FIG. 14. The lamination process and the formation of the medicament layer may be subject to variations to take advantage of manufacturing processes and to produce various embodiments of the medicament layer, as may be desired. The layers of the applicator panel may be co-fed to a pattern roller such that the electrodes and medicament cells are aligned. The roller hermetically bonds the layers together. The resultant multi-layer film of the electrode layer and medicament layer provides an array of isolated iontophoretic cells 28 that may be controlled individually by activation of the electrodes 26.

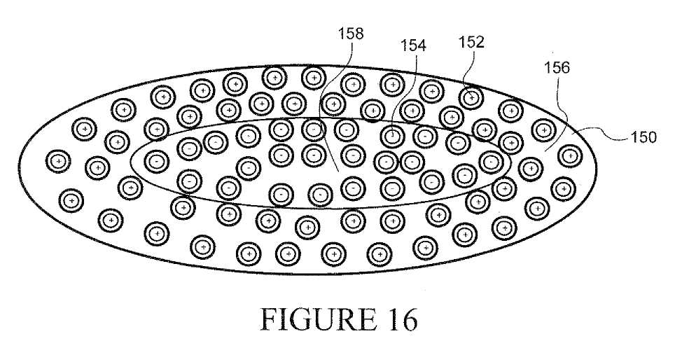

FIG. 16 is a schematic diagram of a front view of an exemplary array 150 of electrodes for the electrode layer. The electrodes in the array include anodic (+) electrodes 152 and cathodic (−) electrodes 154. Anodic electrodes drive medication by applying a net positive charge to the cell. In contrast, cathodic electrodes drive medication by applying a net negative charge to the cell. Certain medications may be best delivered by cathodic electrodes and other medications may be best delivered by anodic electrodes.

A combination of anodic and cathodic electrodes and associated medicament cells may be incorporated in an applicator panel to allow a combination of drugs to be included in the application and delivered simultaneously to the treatment site of a patient. The anodic electrodes 152 may be arranged in an outer border region 156 of the panel 150 and the cathodic electrodes 154 may be arranged in a center region 158 of the panel.

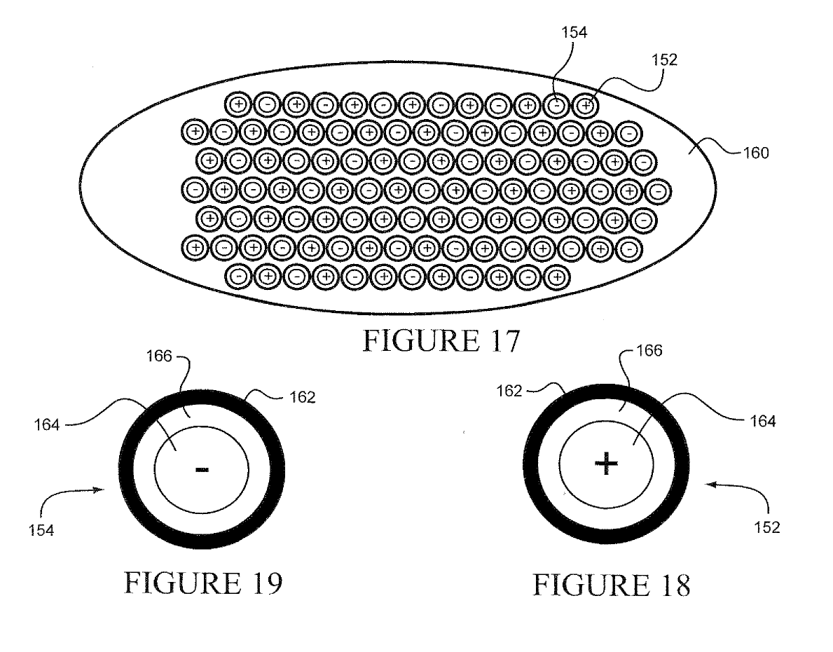

FIG. 17 shows an alternative electrode panel 160 in which the cathodic electrodes 154 are intermingled with the anodic electrodes 152. FIGS. 16 and 17 show patterns of delivery electrodes (the active electrodes) and return or counter electrodes, e.g., neutral electrodes, on the same side of the flexible substrate of the electrode layer. The neutral electrode may surround the anodic or cathodic electrode as shown in FIGS. 18 and 19 or may be arranged proximate to one or more of the anodic or cathodic electrodes, such as by being positioned to be next to the intersections of ribs or ridges of the medicament layers.

Each electrode 152, 154 may be separated from other electrodes in the electrode layer by an insulating, adhesive border. The anodic and cathodic delivery electrodes may be intermingled with anodic electrodes, surrounded by cathodic electrodes or (or the other way around) to provide localized delivery of two or more drugs from the same applicator panel.

From the electrodes and medicament layer, the drug delivery path follows lines of electromagnetic flux from the delivery electrode to the return electrode. The farther away the return electrode is placed from the delivery electrode, the deeper the drug penetrates into the skin. Furthermore, discrete and electrically isolated cells allow the power supply to provide both current polarities to the cells enabling anodic and cathodic delivery at the same time. This in turn allows the delivery of cationic and anionic actives from the same applicator panel and at the same time.

FIGS. 18 and 19 are schematic diagrams of an anodic electrode 152 and a cathodic electrode 154, respectively. A neutral electrode 162, such as establishing a connection to an electrical ground state, may be shaped as a ring to be aligned with the perimeter of a corresponding medicament cell in a medicament layer adjacent to the electrode layer. The neutral electrode 162 may, when adjacent the medicament layer, overlie the ridge section surrounding a cell. The neutral electrode is connected to the distribution circuit on the electrode layer and thereby to the power source and controller. The neutral electrode 162 surrounds and is specific to an active electrode 164, which may be cathodic or anodic. The active electrode is also coupled to the distribution circuit and the power source and controller. Anodic electrodes are connected to a positive voltage source and cathodic electrodes are connected to a negative voltage source. The electrode layer may be a lamination of sub-layers including an anodic electrode layer, a cathodic electrode layer and a neutral electrode layer. These electrode sub-layers include conductive pathways to a connector on the applicator panel that couples to the power source and controller.

Differing drugs may be best suited for delivery by electrodes having different polarities, e.g., cathodic or anodic. The delivery of the medicaments requiring differing polarity for iontophoretic delivery is addressed by providing cathodic and anodic electrodes in electrode layer as is appropriate for the medicaments to be delivered. The counter electrode may be ‘neutral’ with a positive supply coupled to the cathodic electrodes 154 for those cationic drugs requiring a positive active electrode and a negative supply coupled to anodic electrodes 152 for those anionic drugs requiring a negative electrode.

Electrical current flows from the cathodic or anodic electrode 154, 152, into the skin and returns to the surrounding neutral electrode 162. An annular insulation ring 66 separates the outer neutral 162 and the inner active electrode 164. The depth into the skin to which the current flows and correspondingly to which medicament is delivered into the skin depends primarily on the width of the insulation ring 64. In designing the electrode layer, the width of the insulation ring 64 may be selected to achieve a desired depth of medicament delivery, such as to deliver the medicament to the treatment site.

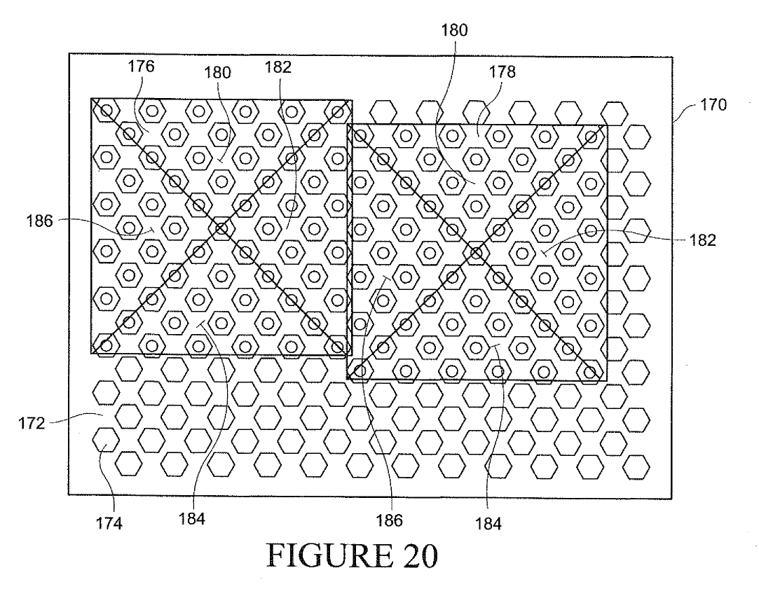

FIG. 20 is a schematic diagram showing a plane view of an exemplary applicator panel 170 having a medicament layer 172 with an array of medicament cells 174. Overlying and bonded to the medicament layer are an electrode layer that includes multiple electrode panels 176, 178. The electrode panels 176, 178 may be rectangular and arranged side-by-side in pattern as shown in FIG. 17, in a concentric arrangement as shown in FIG. 16 or the electrode panels may be superimposed such that the electrodes are intermingled together with respect to the medicament cells. Each panel may have a surface array of three (3) centimeters squared (cm²). The power supply and controller may be programmed to dispense medicament from one or multiple sub-groups of the medicament cells or from all of the medicament sub-layers of medicament layer to a patient.

The panels of electrodes 176, 178 may be segmented into zones of electrodes, wherein each zone activates medicament cells having a different type of medicament than the cells corresponding to the other zones of electrodes. For example, each electrode panel may be segmented into triangular zones 180, 182, 184 and 186 of different grouping of electrodes. The electrodes in each zone are grouped together and are activated as a group. The electrodes of each group/zone are electrically isolated from the electrodes from other zones/groups. Each of the zones of electrodes is superimposed over a corresponding medicament sub-layer of the medicament layer. Each sub-layer may have cells with a drug or other medicine that is different than the drug or medicine in the other sub-layers. When the electrode panel is laminated with a medicament layer 172 having multiple sub-layers, one group of electrodes 180 may be aligned with active medicament cells 174 on a first medicament sub-layer, a second electrode group 182 may aligned with active medicament cells on a second medicament sub-layer, a third electrode group 184 may be aligned with active medicament cells on a third medicament sub-layer, and a fourth electrode group 186 may be aligned with active medicament cells on a fourth medicament sub-layer.

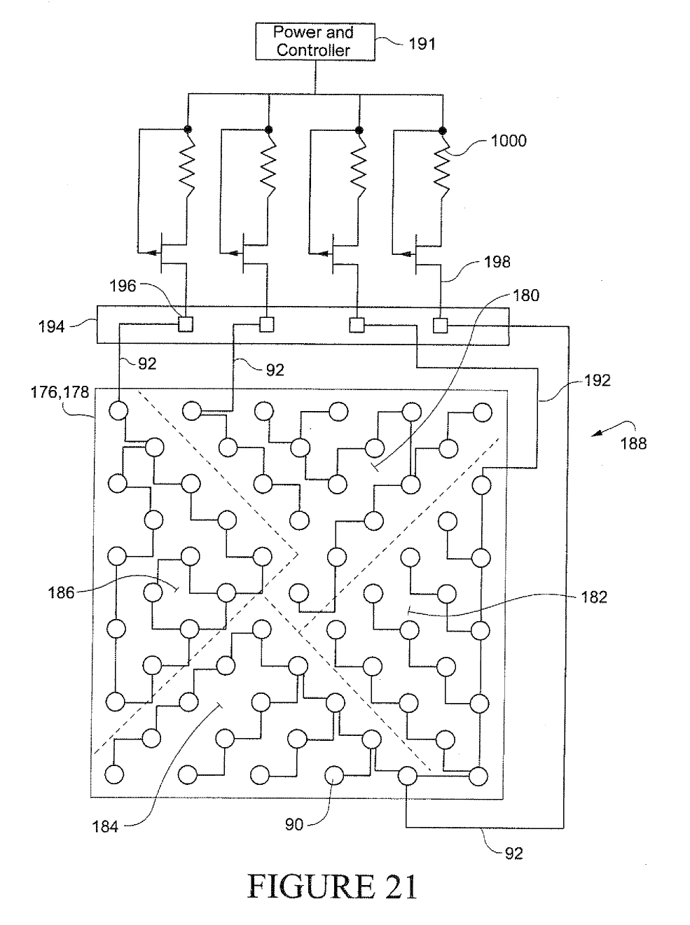

FIG. 21 is schematic diagram of a distribution circuit 188 for one of the electrode panels 176, 178 each having zones of electrodes 180, 182, 184 and 186. The distribution circuit 188 may be arranged on a side of the electrode panel 176, 178 opposite to the side on which the electrodes 190 are mounted. The distribution circuit 188 provides electrical connections between the electrodes and the power supply and controller 191. The electrodes for each zone are interconnected by the distribution circuit. For example, each of the electrodes 190 in zone 184 is interconnected by conductive lines from the distribution circuit. The electrodes from one zone, e.g., zone 184, are not electrically connected to the electrodes in another zone, e.g., zones 180, 182 and 186. A separate connection conductive line or wire 192 may extend from the portion of the distribution circuit connecting the electrodes in one zone, e.g., 184, to a connector 194 that has a separate connection 196 for each of the zones of electrodes 180, 182, 184 and 186. A group of conductive lines, e.g., wires, 198 connects connections 196 for each zone of the electrical panels to the power supply and controller 191.

Each zone of electrodes 180, 182, 184 and 186 covers a relatively small surface area on the skin, e.g., about 1 cm². The current flowing to each zone may be influenced by the contact area between the zone and the skin and the impedance of the contacted skin, e.g., psoriatic skin tissue has lower impedance than does normal skin tissue. A low impedance area of the skin could result in an excessive flow of current that could burn the skin. A current limiting circuit 1000, such as a current limiting diode or constant current source, prevents excessive levels of current flowing to any one of the zones of an electrode panel. Each individual diode or constant current source 1000 may be interrogated by the controller by measuring the voltage at the source resistor. The controller may adjust the current applied to different zones of the electrodes based upon the measured voltages that indicate areas of low impedance.

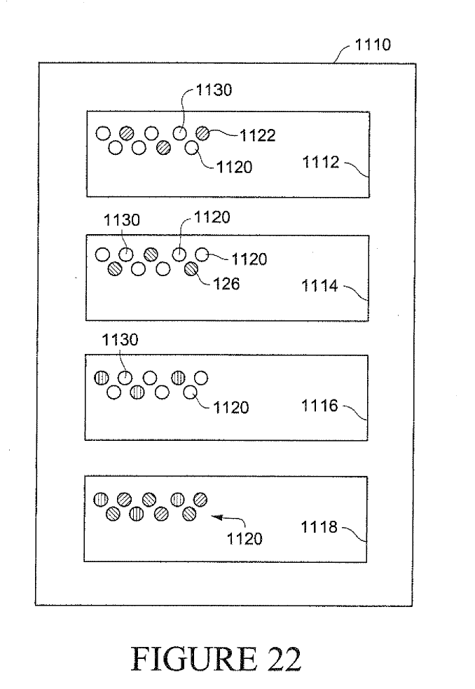

FIG. 22 is an exploded view 1110 multiple sub-layers of a medicament layer 1118 having multiple medicament sub-layers 1112, 1114 and 1116. These layers are superimposed on each other in a lamination process to form the medicament layer 1118. Each medicament sub-layer includes an array of medicament cell locations 1120. In each sub-layer, a sub-set of the medicament cell locations may be filled with medicament. For example, the medicament sub-layer 1112 may include cells 1122 having a first type of drug or medicament, sub-layer 1114 may include cells 1126 having a second type of drug or medicament, and sub-layer 1116 may include cells 1128 with a third type of drug or medicament. Each sub-layers may have openings at those cell locations 1120 in which the sub-layer has no medicament. Preferably, the cells 122, 126 and 128 having medicament in the sub-layers overlap openings 130 in the other sub-layers. After the cells 1122, 1126 and 1128 are loaded with medicament, the sub-layers are superimposed to form an array of cells 1120 in which the medicament cells containing a drug or medicament do not overlap. Cells containing a drug in one layer are aligned with open holes in the other layers so that the cells with drugs abut against the skin when the applicator pad is placed on the skin and the drug flows from these cells directly to the skin when current is applied to the cells.

Each sub-layer 1112, 1114 and 1116 is “addressable” each by a different group of electrodes in the electrode layer. By addressing the sub-layers, medicament may be selectively delivered to the treatment site from just one sub-layer, multiple sub-layers or all sub-layers. Further, the addressing scheme may allow the delivery of medicament to be over a period of time by sequentially addressing the sub-layers over the period of time. The controller may be programmed such that the device delivers one or more the different drugs in the medicament sub-layers, depending on a selection made by the user of drugs to be delivered or depending on an electronically readable prescription prepared by a physician. The housing for the power supply and controller may include a user input (see applicator switch 121 in FIG. 11) to select the drugs to be delivered or a bar code reader (or other electronic reading device) to read the drugs to be delivered based on a prescription passed over the reader. Alternatively, the multiple sub-layers in a medicament layer may each include different dosages of the same drug.

The electrodes of the electrode array laminated over the medicament layer 1118 may be connected such that a first group of electrodes are aligned with the first group of cells 1122, a second group of electrodes are aligned with a second group of cells 1126 and a third group of electrodes are aligned with the third group of cells 1128. Each group of electrodes is separately controlled by the controller. The controller may be programmed to apply current to the electrodes corresponding to in just one of the sub-layers 1112, 1114 and 1116, to a plurality but less than all of the sub-layers or to all of the sub-layers. The user may enter through a user input device personal information, such as gender and weight. With that information, the controller may automatically determine the proper dosage of the drug and which of the medicament sub-layers should be activated to deliver the appropriate dosage.

The number of medicament sub-layers in the medicament layer and the number of electrode panels and zones of electrodes in each applicator pad is a matter of design choice. While an applicator pad may be designed such that each electrode and its corresponding medicament cell may be individually controlled by arranging an appropriate distribution circuit. However, the quantity of cells and electrodes in an applicator pad having an area of 120 cm², such as in a partial facemask applicator, would need to be rather large and may provide an unnecessarily level of fine control over the deliver of medicament. The usage of sub-layers, electrode pads smaller than the applicator pad and electrode zones in each electrode pad allows for a reasonably quantity of control circuits while maintaining a reasonable resolution for managing areas of localized high current density.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. An electrokinetic apparatus to apply medicament to a treatment site of a mammalian user, the apparatus comprising:
a segmented active electrode;
a medicament matrix having one side abutting the segmented active electrode and another side adapted to contact a surface of skin over the treatment site, wherein the apparatus includes at least one current direction control element suppressing transverse current flow through the matrix.

2. An electrokinetic apparatus as in claim 1 wherein the current direction control element is a foam having generally aligned dielectric structures.

3. An electrokinetic apparatus as in claim 1 wherein the current direction control element is a honeycombed structure of the matrix.

4. An electrokinetic apparatus as in claim 1 wherein the current direction control element includes a barrier wall.

5. An electrokinetic apparatus as in claim 4 wherein the barrier wall comprises silicone rubber.

6. An electrokinetic apparatus as in claim 1 further comprising a current limiting device electrically connected in series to each segment of the segmented active electrode.

7. An electrokinetic apparatus as in claim 6 wherein the current limiting device is a diode.

8. An electrokinetic apparatus as in claim 1 wherein each segment of the segmented active electrode is separated by a dielectric gap from other segments.

9. An electrokinetic apparatus as in claim 1 further comprising a cartridge housing the matrix and segmented active electrode, wherein the cartridge is releasable attached to a housing including a power supply and electronic control unit for the active electrode.

10. A method to electrokinetically deliver a medicament to a treatment site in a mammalian user, the method comprising:
applying a first surface of a medicament matrix to a surface on the user;
applying a segmented first electrode to a second surface of the medicament matrix;
applying electrical current to an electrical current path extending through the first electrode segments, medicament matrix, at least partially through the user and to a second electrode;
delivering medicament from the matrix into the nail by electrokinetic transporting the medicament along the current path, and blocking transverse current from one electrode segment of the first electrode to the surface on the user not aligned with the one electrode segment.

11. A method as in claim 10 wherein the first electrode is an active electrode and a second electrode is a counter electrode, wherein current flows from the active electrode to the counter electrode.

12. A method as in claim 10 wherein the blocking of transverse current occurs in the matrix.

13. A method as in claim 10 wherein the matrix includes generally aligned dielectric structures that blocks transverse current.

14. A method as in claim 10 wherein the matrix includes a honeycombed structure that blocks transverse current.

15. A method as in claim 10 wherein the blocking of transverse current is performed by electrically coupling a pair of active and counter electrode segments and isolating each pair from another pair of active and counter electrode segments.

16. A method as in claim 15 wherein the pair of active and counter electrode segments is coupled to a secondary winding of a transformer, and another secondary winding is coupled t the another pair of active and counter electrode segments.

17. An electrokinetic apparatus to apply medicament to a treatment site of a mammalian user, the apparatus comprising:
an active electrode including first and second active electrode segments;
a counter electrode including first and second counter electrode segments;
a medicament matrix having one side abutting the segments of the active electrode and another side adapted to contact a surface of the user over the treatment site, wherein the matrix includes at least one current direction barrier suppressing transverse current flow through the matrix;
a first electrical circuit including the first active electrode segment and the first counter electrode segment, and
a second electrical circuit including the second active electrode segment and the second counter electrode segment, wherein the first electrical circuit is electrically isolated from the second electrical circuit.

18. An electrokinetic apparatus as in claim 17 wherein the surface of the user is a nail of a toe or finger, and the medicament matrix is a porous sheet applied to the nail.

19. An electrokinetic apparatus as in claim 17 wherein the first electrical circuit includes a first secondary winding of a transformer and the second electrical circuit includes a second secondary winding.

20. An electrokinetic apparatus as in claim 17 wherein the first and second secondary windings are electromagnetically coupled to a common primary winding of the transformer.

21. An applicator panel for an electrokinetic delivery system comprising:
an array of electrodes arranged on a flexible substrate and conductive pathways between each of the electrodes and a connector to receive a connection to a electronic control and power source circuit, wherein each electrode includes a center active electrode region and an outer return electrode region proximate to the active electrode region, and
at least one layer having a pattern of medicament cells and network of ribs between the cells, wherein the non-woven layer has a back surface laminated to the array of electrodes and the cells are each aligned with a respective one of the electrodes.

22. The applicator panel of claim 21 further comprising an adhesive matrix on a front surface of the non-woven layer, wherein the adhesive is aligned with the network of ribs, wherein the non-woven layer is sandwiched between the array of electrodes and the adhesive matrix.

23. The applicator panel of claim 21 wherein the at least one non-woven layer has a plurality of sub-layers each of which includes a pattern of medicament cells which are offset from medicament cells in the other sub-layers when the sub-layers are laminated together.

24. The applicator panel of claim 23 wherein each sub-layer includes apertures which align with the medicament cells in the other sub-layers when the sub-layers are laminated together.

25. The applicator panel of claim 23 wherein one of the sub-layers includes a medicament different from a medicament in another of the sub-layers.

26. The applicator panel of claim 21 wherein the array of electrodes includes a plurality of zones of electrodes wherein each zone of electrodes is separately controlled by a controller and power source, and each zone aligns with a different group of the cells.

27. The applicator panel of claim 21 wherein the array of electrodes includes a plurality of separate electrode panels and each panel aligned with a different group of the cells in the at least one non-woven layer.

28. The applicator panel of claim 27 wherein each of the electrode panels includes at least a first zone of electrodes and a second zone of electrodes, wherein the first zone of electrodes in each of the panels are activated by a power source simultaneously and the second zone of electrodes in each of the panels are active by the power source simultaneously and separately of the first zone of electrodes.

29. The applicator panel of claim 21 wherein the electrodes each include a center active electrode, a counter electrode surrounding the active electrode and an insulating ring between the active electrode and counter electrode.

30. An applicator for an electrokinetic delivery system comprising:
an array of electrodes arranged on a substrate and conductive pathways between each of the electrodes and a connector to receive a connection to a electronic control and power source circuit, wherein the electrodes are arranged in at least a first commonly connected group of electrodes and a second commonly connected group of electrodes;
a first medicament layer having a pattern of first medicament cells, wherein the first medicament cells are aligned with the electrodes of the first commonly connected group of electrodes;
a second medicament layer having a pattern of second medicament cells, wherein the second medicament cells are aligned with the electrodes of the second commonly connected group of electrodes, and
an electronic controller controlling the electrokinetic delivery to a treatment site of a patient and underlying the first and second medicament layers, wherein medicament in the first medicament layer is delivered by electrically actuating the first commonly connected group of electrodes and medicament in the second medicament layer is delivered by electrically actuating the second commonly connected group of electrodes.

31. The applicator in claim 30 wherein the pattern of first medicament cells and the pattern of second medicament cells are embossed and the first medicament layer and second medicament layer each comprise a network of ribs between the medicament cells.

32. The applicator in claim 30 wherein the first medicament cells include a first medicament and the second medicament cells include a second medicament different than the first medicament.

33. An applicator panel for an electrokinetic delivery system comprising:
an array of electrodes arranged on a flexible substrate and conductive pathways between each of the electrodes and a connector to receive a connection to a electronic control and power source circuit;
at least one non-woven layer having a pattern of medicament cells and ribs between the cells, wherein the cells are aligned with electrodes of the array;
an adhesive matrix where the adhesive is aligned with the ribs, wherein the non-woven layer is sandwiched between the array of electrodes and the adhesive matrix.

34. The applicator panel as in claim 33 wherein the pattern of medicament cells are embossed on the non-woven layer.

35. The applicator panel as in claim 33 wherein the ribs are ridges raised on a side of the non-woven layer facing the array of electrodes.

36. The applicator panel as in claim 35 wherein the ridges do not rise above a front surface of each of the cells on a side of the non-woven layer opposite to the array of electrodes.

37. The applicator panel as in claim 33 wherein the ribs form a network of rows and columns between the cells.

38. A method to form an applicator panel for an electrokinetic delivery system comprising:
embossing a pattern of medicament cells and ridges on a non-woven layer;
infusing a medicament into the medicament cells;
forming an electrode layer by applying an array of electrodes on a front side of an electrode layer substrate and forming an electrical distribution circuit on a back side of the electrode layer substrate, and
securing the front side of the electrode layer substrate to a back side of the non-woven layer, wherein the electrodes are aligned with the medicament cells.

39. The method of claim 38 further comprising forming a first sub-layer and a second sub-layer of the non-woven layer, wherein each of the sub-layers includes a separate group of the medicament cells and the group of the medicament cells in the first sub-layer is non-overlapping with the group of the medicament cells in the second sub-layer.

40. An array of electrodes in an applicator panel for an electrokinetic delivery system including a medicament layer having an array of medicament cells, each of the electrodes comprising:
a center electrode region aligned with one of the medicament cells;
a non-conductive region surrounding the center electrode, and
a neutral return electrode region encircling the annular adhesive seal.

41. The array of electrodes in claim 40 wherein the non-conductive region of each electrode is a ring.

42. The array of electrodes as in claim 40 wherein the center region in each of a first plurality of the electrodes is a cathodic center region coupled to a source of positive voltage and the center region in each of a second plurality of the electrodes is an anodic center region coupled to a source of negative voltage.

43. The array of electrodes as in claim 42 wherein the first plurality of the electrodes with the cathodic center regions are intermingled with the second plurality of the electrodes with the anodic center regions.

44. The array of electrodes as in claim 42 wherein the first plurality of the electrodes with the cathodic center regions are arranged in a group separate from the second plurality of the electrodes with the anodic center regions.

45. The array of electrodes as in claim 42 wherein the non-conductive region of each electrode includes an adhesive to bind the electrode to the medicament layer.

46. The array of electrodes as in claim 42 wherein a width of the non-conductive ring determines a depth of medicament delivery into skin, and the width is selected to achieve a desired depth of medicament delivery.

47. The array of electrodes as in claim 42 further comprising a current regulation circuit electrically coupled to each electrode.

48. The array of electrodes as in claim 47 wherein there is one of the current regulation circuits for each electrode.
